(19) United States
(12) Patent Application Publication
(10) Pub. No.: US 2022/0383896 A1
(43) Pub. Date: Dec. 1, 2022

(54) SYSTEM AND METHOD FOR COLLECTING BEHAVIOURAL DATA TO ASSIST INTERPERSONAL INTERACTION

(71) Applicant: Realeyes OÜ, Tallinn Harju (EE)

(72) Inventors: Elnar HAJIYEV, Tallinn (EE); Martin SALO, Tallinn (EE)

(21) Appl. No.: 17/774,991

(22) PCT Filed: Nov. 10, 2020
(86) PCT No.: PCT/EP2020/081673

§ 371 Date: May 6, 2022

(30) Foreign Application Priority Data
Nov. 12, 2019 (GB) 1916435.9

(51) Int. Cl. G10L 25/72; G06V 40/20; G10L 25/63

(52) U.S. Cl. CPC G10L 25/72; G06V 40/20; G10L 25/63

(57) ABSTRACT

A system for capturing behavioural data in order to influence an interpersonal interaction. In one aspect, the system assists n the training of an entity whose role is to engage in such interpersonal interactions. In this aspect, the collected information may be used to judge performance, and/or adapt or improve approach to future interactions, In another aspect, the system may assist with a live interaction, i.e. to provide feedback in an ongoing conversation. The system comprises a wearable device having (i) a data collection device configured to capture behavioural data during the interpersonal interaction, (ii) a microphone configured to capture audio data during the interpersonal interaction, and (ill) an analysis module arranged to extract emotional state information and content data, and use that extracted data to evaluate an interaction quality metric to obtain an interaction score for the interpersonal interaction.

### FIELD OF THE INVENTION

The invention relates to a computer-implemented system and method for collecting behavioural data from one or more participants in an interpersonal interaction (e.g. a conversation, meeting, etc.) for the purpose of assisting the interaction, e.g. improving its effectiveness or influencing its outcome. The interaction may be a conversation between a salesperson and potential customer, e.g. in a shop or other sales environment. The salesperson may be a human or a social robot.

In one example, the collected behavioural data is used to generate feedback information that is passed back to a participant in the interaction, e.g. to provide information about the emotion state or engagement of other participants, or to provide suggestions for improving the interaction. In another example, the collected behavioural data is used to assess the performance of a participant in the conversation. This assessment may be used as a training tool, e.g. to monitor the development of the participant. Or it may be used to determine other actions, e.g. recommend a break, etc.

### BACKGROUND TO THE INVENTION

It is known to measure emotional state data in a passive manner, e.g. by collecting data indicative of a user's behavioural or physiological characteristics, e.g. while they consume media content or engage in certain activities. For example, facial responses can be used as passive indicators of experienced emotional state. Webcam video acquisition can be used to monitor facial responses, by capturing image frames as a piece of media content is consumed by a user. Emotional state can therefore be captured through the use of webcams, by processing video images.

Physiological parameters can also be good indicators of experienced emotional state. Many physiological parameters are not consciously controllable, i.e. a consumer has no influence over them. They can therefore be used to determine the true emotional state of a user when engaged in a certain activity. Examples of physiological parameters that can be measured include voice analysis, heartrate, heartrate variability, electrodermal activity (which may be indicative of arousal), breathing, body temperature, electrocardiogram (ECG) signals, and electroencephalogram (EEG) signals.

Emotional state information measured in this way has been shown to correlate with commercial performance, for example sales lift. This information has therefore been used as a tool to target advertising as a user interacts with a computing device in order to maximise its effectiveness.

The behavioural characteristics of a user may manifest themselves in a variety of ways. References to “behavioural data” or “behavioural information” herein may refer to visual aspects of a user's response. For example, behavioural information may include facial response, head and body gestures or pose, and gaze tracking. In practice, it can be desirable to use a combination of raw data inputs comprising behavioural data, physiological data and self-reported data in order to obtain emotional state information. A combination of raw data from two or three of the sources mentioned above may be useful in identifying “false” indicators. For example, if emotional state data derived from all three sources overlaps or is aligned, it gives more confidence in the obtained signal. Any inconsistency in the signal may be indicative of a false reading.

### SUMMARY OF THE INVENTION

At its most general, the present invention proposes a system in which behavioural data is captured and used to influence an interpersonal interaction. The discussion below presents two aspects. In a first aspect, the influence is to assist in the training of an entity whose role is to engage in such interpersonal interactions. This entity may be a person (e.g. a salesperson or company representative) or a social robot configured to engage in interpersonal interactions. In this aspect, the collected information may be used to judge their performance, and/or adapt or improve their approach to future interactions. In a second aspect, the influence may be on a live interaction, i.e. to provide assistance in an ongoing conversation. The assistance may be simply in increasing awareness, or may be an active tool, e.g. that makes suggestions for influencing the direction of the conversation.

According to the invention, there is provided a system for collecting behavioural data during an interpersonal interaction, the system comprising: a wearable device comprising: a data collection device configured to capture behavioural data during the interpersonal interaction; a microphone configured to capture audio data during the interpersonal interaction; an analysis module arranged to: extract emotional state information from the behavioural data; extract content data from the audio data; evaluate, using the emotional state information and content data, an interaction quality metric to obtain an interaction score for the interpersonal interaction.

As mentioned above, the behavioural data may relate to visual aspects of a participant's response to the interpersonal interaction. For example, the behavioural data may include facial response, head and body gestures or pose, and gaze tracking. The data collection device may comprise any suitable device for capturing such data. For example, the data collection device may comprise an imaging device configured to capture image data during the interpersonal interaction.

The interpersonal interaction may be between a first participant and a second participant. In one use scenario, the first participant may be seeking information or a service from the second participant. The second participant may wear the wearable device to capture the image data and audio data concerning the interaction.

With this system, the interpersonal interaction can be monitored either in real time or after the event. The interaction score may be a quantitative value, e.g. on a scale of 1-10, where 1 indicates a very poor interaction and 10 indicates a very good interaction. Or the interaction score may be one of a number of discrete indicators, e.g. “poor”, “adequate”, “good”.

The interaction quality metric may be any suitable entity for transforming the collected data into the interaction score. For example, the interaction quality metric may be a classifier or other algorithm that uses the collected data as an input and outputs the interaction score. The interaction quality metric may alternatively be a rule-based decision tree, e.g. in which properties of the collected data are compared with thresholds to determine a category or value for the interaction score.

The wearable device may take any suitable form. For example, it may be a badge, armband, headgear (e.g. hat or headband), glasses, neckwear (e.g. lanyard or necklace).

The analysis module may be any suitable computing device, i.e. device capable of processing the capture image data and audio data. The analysis module may be provided in the wearable device or in a separate device, located remotely from the wearable device. In another example, the analysis module may be distributed between the wearable device and a remote device. In this case, the analysis module may extract the emotional state information and the content data at the wearable device, whereas the evaluation of the interaction quality metric occurs remotely. This arrangement may be advantageous because it helps preserve a participant's privacy, e.g. because it does not require image data or audio data to be transmitted away from the wearable device, without introducing a high processing burden at the wearable device.

The analysis module may be further arranged to determine an interaction type for the interpersonal interaction. The interaction type may be based on the content data. The interaction type may be used as a label to assist in identifying and categorising interactions. As such it can assist in training, e.g. because a participant's interaction score can be assessed based on interaction type, and in determining feedback, e.g. by enabling comparison with other similar interactions, as explained below.

The interaction type may be selected from a list of predetermined interaction types. The interaction type may have a vector structure, in which a probability is associated with each of the predetermined interaction types. The selection or probability allocation may be done automatically, e.g. using a classifier or other suitable algorithm configured to map content data to one or more interaction types. The classifier may use the content data only, or may use a combination of the content data and the emotion state information.

The analysis module may be further arranged to generate and store an interaction record for the interpersonal interaction. The interaction record may be stored in a database, e.g. remotely from the wearable device. Alternatively, the interaction record may be generated and stored on the wearable device, and transferred to a repository later.

The interaction record may be a data structure that associates the interaction type with the interaction score and an emotional state profile indicative of variation in the emotion state information during the interpersonal interaction. The analysis module may be arranged to measure change in emotional state with time to generate the emotional state profile. The interaction record may thus be indicative of the evolution of the interaction. The interaction score itself may be a time-varying parameter. For example, the interaction score may be evaluated at intervals, e.g. regular Intervals or at certain predetermined points, to provide an indication of relative change in the interaction.

The interaction record may further comprise a plurality of content tags indicative of the content data. The content tags may be key words or phrases extracted or otherwise from the audio. The content tags may be indicative of the subject matter of the conversation. Each content tag may be associated with a time frame of the interaction, so that the evolution of the content during the interaction is recorded.

The emotional state profile may have similar time frames, which means that the content tags may be associated with certain emotions or certain changes in emotional state. This may enable patterns between content and emotional state to be recognised in the interaction quality metric.

The interaction record may further comprise participant identification data. For example, the image data and/or audio data may be used to generate an identity label for each participant in the interaction. The identity label may be extracted from the image data and/or audio data in a manner that preserves the participants privacy, e.g. by avoiding storage of raw image or audio data. The participant identification data may be used to recognise patterns and/or preferences in the manner that a given participant engages in interactions.

The system may further comprise a database arranged to store a plurality of interaction records corresponding to a plurality of interpersonal interactions. The interactions may be between different participants. Each interaction record may be configured as set out above, e.g. to include any one or more of an interaction type, content tags, emotional state profile, participant identifier(s), and interaction score. The database may thus store a repository of interaction records, which form a rich data source that can be used to obtain recommendation or, with suitable labelling, train classification algorithms.

The interaction score obtained by evaluating the interaction quality metric may permit interaction records to be ranked, e.g. to help obtain recommendations, as discussed below.

The interaction score obtained by evaluating the interaction quality metric may be statistical, e.g. based on a comparison with one or more norms extracted from the database.

The system may further comprise a feedback generation module arranged to: determine a recommendation for improving the interaction score for the interpersonal interaction; generate a feedback instruction corresponding to the recommendation; and output the feedback instruction. The recommendation may be determined after the interaction has ended, whereby the feedback may relate to training of a participant to try to improve their performance in future interactions. Alternatively or additionally, the recommendation may be determined in real time as the interaction is progressing, whereby the feedback may relate to improving an outcome of the current interaction.

The recommendation may be obtained, e.g. extracted or derived, from stored interaction records, i.e. the repository discussed above. The recommendation may be determined using a statistical or rule-based procedure. For example, the feedback generation module may be arranged to look up one or more interaction records that have a matching interaction type, similar content tags and better interaction score than the current interaction. The recommendation may be content-based, e.g. to introduce a certain topic into conversation or emotion-based, e.g. to suggest an action that encourage certain emotional response.

The system may further comprise a feedback communicator arranged to receive the feedback instruction from the analysis engine. The feedback communicator may comprise a second wearable device, e.g. worn by one of the participants in the interaction. For example, the second wearable device may be an ear piece. Alternatively or additionally the feedback communicator may comprise a portable computing device, e.g. smartphone, tablet computer or the like.

The analysis module may comprise separate units for processing the image data and audio data. For example, the analysis module may comprise an image analyser module arranged to extract emotional state information from the image data captured by the imaging device. The image analysis module may operate in any known manner for obtaining emotional or mental state data from a facial image of a participant, e.g. using techniques disclosed in US 2017/0105668 A1, for example.

The analysis module may comprise a speech recognition module configured to extract the content data from the audio data captured by the microphone. The speech recognition module may utilise a natural language processing tool to derive the content data from the captured audio data.

The analysis module may utilise both the audio data and the image data to extract the emotional state information. For example, the analysis module may include a first classifier configured to map the audio (and in particular tone information in the audio data) in to emotional state information. The analysis module may include a second classifier configured to map a facial image into the emotional state information. The outputs from the first and second classifiers may be combined or used for validation. Alternatively, the analysis module may comprise a combined classifier that receives both the audio data and the image data as an input to be mapped to emotional state information. The combined classifier may also use the content data as an input.

The analysis module may comprise an analysis engine configured to evaluate, using the emotional state information and content data, the interaction quality metric to obtain the interaction score. As mentioned above, the analysis engine may be located remotely from the wearable device. The wearable device may comprise a transceiver configured to wirelessly transmit the emotional state information and the content data to the analysis engine. Alternatively, the analysis engine may be provided on the wearable device.

The system may comprise an intermediate computing device arranged in wireless communication with the wearable device. The analysis module may be provided on the intermediate computing device. The wearable device may thus comprise a transceiver configured to wirelessly communicate the image data and audio data to the intermediate computing device, e.g. using a short range network (e.g. Bluetooth®) or over a local area network (e.g. WiFi).

The wearable device may comprise a processor and a memory having software instructions stored thereon, wherein the analysis module comprises one or more software modules which run by execution of the software instructions by the processor.

As discussed above, the image data is of a first participant in the interpersonal interaction. For example, it may comprise a series of facial images of the first participant. The wearable device may be configured for a second participant in the interpersonal interaction. The second participant may be a human or a social robot, e.g. an interactive digital assistant. The interaction score and interaction record may provide a means for monitoring the performance of a robot participant. In some examples, this monitoring may involve comparing interaction profiles from a robot with interaction profiles of a human.

In another aspect, the invention may provide a computer-implemented method of collecting behavioural data relating to an interpersonal interaction between a first participant and a second participant, the method comprising: collecting behavioural data of the first participant using a wearable device worn by the second participant, wherein the behavioural data comprises an image data stream and an audio data stream captured during the interpersonal interaction; extracting an emotional state information stream from the image data stream; extracting a content data stream from the audio data; evaluating, using the emotional state information stream and content data stream, an interaction quality metric to obtain an interaction score for the interpersonal interaction.

The method may include determining an interaction type for the interpersonal interaction based on the content data;

and generating an interaction record for the interpersonal interaction. As explained above, the interaction record may be a data structure that associates the interaction type and interaction score with an emotional state profile that is indicative of variation in the emotion state information during the interpersonal interaction. The interaction record may further comprise any one or more of: a plurality of content tags indicative of the content data; and participant identification data.

The method may include storing, in a database, a plurality of interaction records from multiple interpersonal interactions. In one example, the method may include aggregating a plurality of interaction records, e.g. that Involve the second participant. The interaction records may be normalised before aggregation. The aggregated interaction records may be used to obtain norms indicative of the performance of the second participant across a plurality of interactions.

The method may further comprise determining a recommendation for improving the interaction score for the

Interpersonal interaction; generating a feedback instruction corresponding to the recommendation; and outputting the feedback instruction. The step of determining the recommendation may comprise: searching for one or more interaction records in database that have corresponding content data and a higher interaction score; and identifying differences between the current interaction and the one or more interaction records, wherein the recommendation is based on differences associated with an improved emotional state or interaction score. Searching for interaction records with corresponding content data may include evaluating a correlation between content data in the current interaction and in one or more interaction records having a matching interaction type in the database.

The method may include comparing the performance of different second participants, e.g. to influence decision on who should assist with future interactions. For example, the method may obtaining a first plurality of interaction records from multiple interpersonal interactions in which the second participant is a human; obtaining a second plurality of interaction records from multiple interpersonal interactions in which the second participant is a social robot; comparing the first plurality of interaction records with the second plurality of interaction records to determine a performance metric for the human and the social robot; and selecting either the human or the social robot to participate in a future interaction based on the determined performance metric. The comparison may be based on interaction score. Although this example compares a human participant with a robot participant, it is equally possible to compare different human or different robots.

### BRIEF DESCRIPTION OF THE DRAWINGS

Embodiments of the invention are discussed in detail below with reference to the accompanying drawings, in which:

FIG. 1 is a schematic diagram of a behavioural data collection and analysis system that is an embodiment of the invention;

FIG. 2 is a schematic diagram of a wearable data collection tag that may be used in the invention;

FIG. 3 is a schematic diagram of an analysis system that may be used in the invention;

FIG. 4 is a flow diagram of a data collection method that is an embodiment of the invention; and

FIG. 5 is a schematic representation of an interaction record data structure that can be used in embodiments of the invention.

### DETAILED DESCRIPTION; FURTHER OPTIONS AND PREFERENCES

Embodiments of the invention relate to a system and method of collecting and utilising behavioural data during an interpersonal interaction.

FIG. 1 is a schematic diagram of a complete data collection and analysis system 100 that is an embodiment of the invention. It can be understood that the system in FIG. 1 illustrates components for performing collection and analysis of data, as well as for subsequent use of that data, e.g. in providing direct feedback for the interaction or in assessing the performance of a participant in the interaction. In other examples separate systems having these functionalities may be provided.

Herein the term “interpersonal interaction” is intended to encompass any type of live social intercourse between individuals. Each individual, who may be referred to as a participant in the interaction, may be human or may be a social robot designed to simulate a human. The individual may be a digital robot assistant, for example. The interaction may be a one-to-one interaction, e.g. a conversation between two people, or may be a multi-person interaction, such as a meeting, seminar, or the like. The system may be configured for use with certain types of interaction, e.g. conversations that occur in certain contexts. For example, the system may be used in a commercial setting, such as a department store or showroom, where the participants in the interaction are a customer (potential purchaser) on the one hand, and a salesperson or store representative on the other hand.

The example shown in FIG. 1 is in the context of a conversation between two people. A first participant 101 may be referred to below as a consumer or customer. A second participant 103 may be a salesperson or robotic store assistant. An aim of the system may be to provide feedback to the second participant 103 to improve or maximise the effectiveness of interaction with the first participant 101. The effectiveness may be set or measured relative to a certain objective, e.g. ensuring that the first participant reaches a positive emotional state by the end of the interaction, or that the interaction leads to a desired outcome or action, such as a sale.

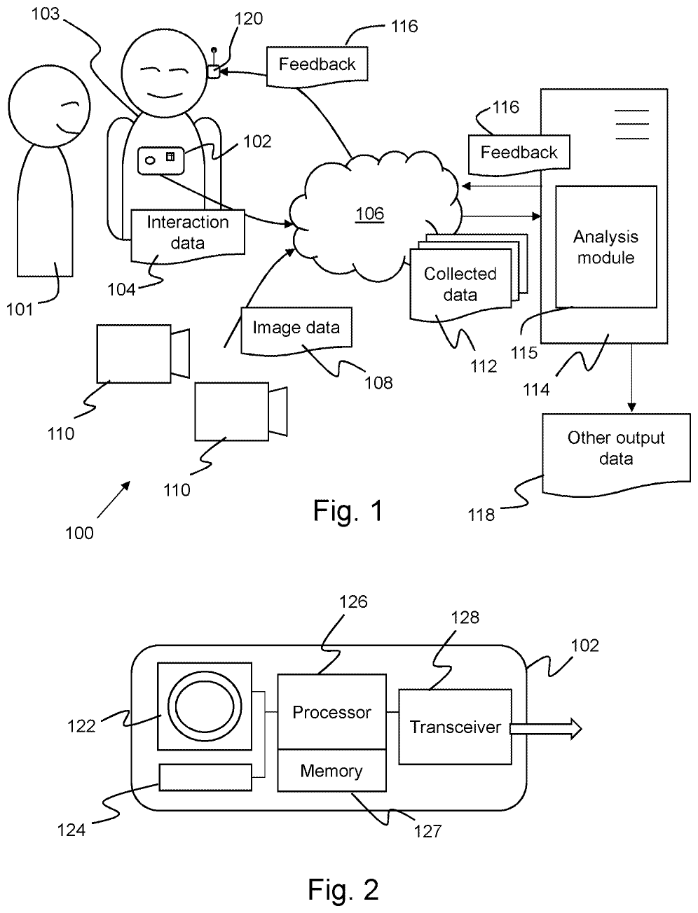

The system 100 is provided in a networked computing environment, where a number of processing entities are communicably connected over one or more networks. In this example, the system 100 comprises a wearable data collection device 102 that is arranged to collect (e.g. record) behavioural data. For example, wearable data collection device 102 may comprise behavioural data capture apparatus, such as a camera and microphones for capturing audio and image data relating to the interaction, as discussed in more detail below with respect to FIG. 2. The wearable device 102 is worn by one of the participants in the interaction. In this example, it is worn by the second participant 103. The wearable device 102 may resemble a name badge, for example.

The system 100 may also comprise one or more participant sensors units (not shown), such as additional wearable devices (on either participant) for collecting physiological information from a participant during the interaction. Examples of physiological parameters that can be measured include voice analysis, heartrate, heartrate variability, electrodermal activity (which may be indicative of arousal), breathing, body temperature, electrocardiogram (ECG) signals, and electroencephalogram (EEG) signals.

The wearable device 102 is communicably connected over a network 106 to an analysis server 114. The wearable device 102 operates to send interaction data 104 over the network 106 for analysis or further processing at a remote device, such as analysis server 114. The interaction data 104 may comprises the collected raw data and/or behavioural data derived from the collected raw data by an analysis module on the wearable device itself.

As mentioned above, references to “behavioral data” or “behavioral information” herein may refer to any collected information about the user's response, e.g. visual aspects of a user's response or physiological data. For example, behavioral information may include facial response, head and body gestures or pose, and gaze tracking.

In one example, the interaction data 104 sent to the analysis server 114 may include a user's facial response, e.g.

in the form or a video or set of images captured of the user during the interaction. The interaction data 104 may also Include an audio recording of the interaction. In other examples, the raw data (audio and image) may not be transmitted in the interaction data 104. Instead, to maintain privacy, the raw data may be analysed at the wearable device 102 to extract anonymised information therefrom. For example, the wearable device 102 may be configured to extract emotional state information from the raw data. For example, emotional state information may be extracted from facial images in the image data and/or variation in tone in the audio data. The interaction data 104 may include information indicative of the evolution of emotional state of a participant during the interaction without sending any images of that participant. The wearable device may also be configured to extract content data from the audio signal, e.g. using a speech analysis module. The interaction data 104 may thus include information indicative of the content of the interaction without sending an actual recording of the interaction.

The behavioural data collected for the interaction may not come solely from the wearable device 102. There may be other behavioural data collection devices in the vicinity of the interaction. For example, if the interaction occurs in a shop or showroom, there may be cameras 110 (e.g. CCTV or the like) that capture image data 108 (and/or audio data) of the interaction. The cameras 110 may be connected over the network to supply the image data 108 to the analysis server 114. The analysis server 114 may thus effectively receive a data stream comprising collected data 112 from a plurality of sources, where the collected data 112 comprises behavioural data relating to the interaction.

The collected data 112 sent to the analysis server 114 may also include physiological data obtained from one or more participants during the interaction. The physiological data may be transmitted directly by a respective wearable device, or the wearable device that records the physiological data may be paired with the wearable device 102, which is arranged to receive and send on the physiological data.

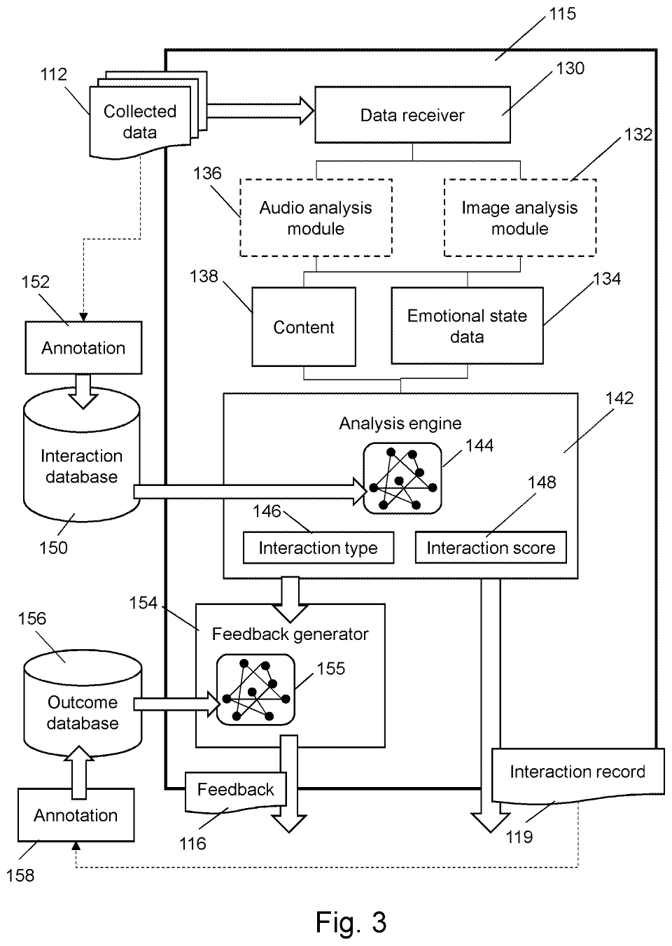

The analysis server 114 is a computing device having a processor and memory (not shown) configured to execute software instructions that cause it to carry out the functions of an analysis module 115. The tasks performed by the analysis module 115 are discussed in more detail below with respect to FIG. 3.

The analysis module 115 may be configured to use the collected data 112 to monitor the interaction. The monitoring may be performed in a number of ways. For example, the monitoring may comprises determining changes in the emotional state or attentiveness of the first participant 101. In conjunction with information about the content of the interaction, the analysis server may be configured to output information 118 that links performance of the second participant 103 with changes in the mood or engagement of the first participant 101. This information may be recorded, e.g. to assist in training the second participant.

The analysis module 115 may also use the content data to categorise the interaction, i.e. by assigning it an interaction type, e.g. selected from a list of predetermined interaction types. This can facilitate comparison of interactions that are similar to one another.

One aspect of monitoring interactions is an ability to score an interaction based on the captured data. This is done by evaluating, by the analysis module 115, an interaction quality metric. The interaction quality metric may be an algorithm, e.g. a classifier or other suitable mapping function that uses the content data and emotional state information as an input and outputs an interaction score that is indicative of the quality of the interaction.

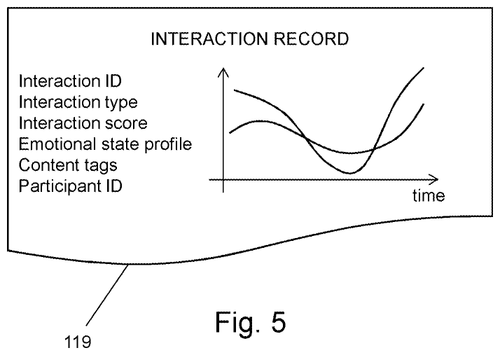

The interaction score may be combined with other information relating to the interaction to generate output information 118. The output information 118 make take the form of an interaction record 119. FIG. 5 shown a schematic representation of an example interaction record data structure. The interaction record 119 may be a data structure having data fields populated with any one or more of an interaction identifier, an interaction type, the interaction score, an emotional state profile, a set of content tags and participant identification data. The emotional state profile may be a data set or graphical indication of variation over time of one or more emotional states of a participant.

The interaction score may also be a time varying entity. For example, the analysis module 115 may be arranged to evaluate the interaction quality metric at certain points during the interaction, e.g. in real time. The interaction score may thus vary through the duration of the interaction.

Another task performed by the analysis server 114 may be to provide feedback 116 to the second participant 103 over the network 106. In one example, the second participant 103 possesses a feedback receiving device 120, which may be an earpiece, smartphone, tablet computer, laptop computer or the like. The feedback receiving device 120 may be configured to display, play back or otherwise notify the second participant 103 of the feedback 116. The feedback 116 may comprise hints or suggestions to assist the second participant 103 to improve the interaction.

The feedback 116 may be generated automatically at the analysis module 115 by comparing one or more features of the interaction record for the current interaction with a plurality of interaction records relating to previous interactions. The analysis module 115 may include a model configured to determine a recommendation based on differences between the interaction record for the current interaction with stored interaction records for previous interactions (e.g. having the same interaction type).

The model may be based on a neural network or similar machine learning technique, that has been trained using behavioural data collected for a plurality of interactions whose performance against the objective is known.

For example, the objective may relate to ensuring that the first participant ends the interaction with a positive emotion state. The analysis of the collected data may provide information about the first participant's current emotional state. In addition to emotional state information, the collected data may also include contextual data, e.g. relating to the information content (subject matter) of the interaction (i.e. what the participants are discussing), and the tone of the discussion (e.g. friendly, aggressive, neutral, etc.). The contextual data is also included in the training set, so the model is able to provide feedback in relation to the context of the interaction, e.g. to suggest a topic to discuss, or a question to ask, in order to steer the interaction towards meeting the objective. For example, if the emotional state of the first participant is ascertained to

Include doubt or a lack of confidence, the second participant may be encouraged to strike up a conversation.

The feedback 116 may be an instruction determined based on a trend in the performance of the second participant 103. For example, if the collected data is indicative of fatigue in the second participant, the feedback 116 may encourage or Instruct them to take a break. In another example, if the trend of the interaction becomes negative, the feedback may Instruct the second participant to request assistance, e.g. by calling a manager. In some cases, the assistance may be automatically called.

In another example, feedback 116 can be generated locally, e.g. by comparing collecting behavioural data with preset thresholds rather than analysing against other stored interaction records. For example, the feedback 116 may encourage the second participant to seek assistance if the emotional state information indicates a downward trend in the emotional state of the first participant.

The provision of feedback 116 may be optional. In one example, the invention may be used purely as a monitoring tool in order to assess and improvement the performance of the second participant in future interactions. In this example, the analysis server 114 may generate output data 118 that is indicative of changes in the first participant's emotional state and/or engagement throughout the interaction. This information may be associated with the contextual data, so that the output data 118 links changes in emotional state or engagement with what was occurring in the interaction.

The output data 118 may be used to benchmark the performance of the second participant 103, or as a means for identifying areas where improvement or additional training is needed.

The possible uses above are just some examples of what can be done with the collected data 112. The wearable device 102 and analysis server 104 may form a networked-enabled platform for the collection and delivery of real time information that links the emotional state of participants in an interaction with the context of that interaction. Such a platform may find uses beyond the retail sector. For example, it may be used to improve situational awareness in other types of interpersonal interaction, e.g. board meetings, lectures or seminars, or the like.

FIG. 2 shows a schematic diagram of a wearable device 102 that may be used in the invention. As mentioned above, the wearable device 102 may resemble a name tag, e.g. to be pinned to the clothing of a participant in the interaction. However, the wearable device need not be limited to this form factor. It may be embodied in headwear, e.g. a hat or cap, or in spectacles or a necklace. All of these examples enable the wearable device 102 to face in a similar direction to the participant, which means it is easy for the participant to direct the wearable device 102 towards other participants in the interaction.

The wearable device 102 comprises a housing for supporting a number of functional modules. For example, the wearable device 102 may comprise a camera 122 (e.g. a webcam or the like) for capturing image data.

The wearable device 102 may further comprise a microphone 124 for capturing audio data.

As mentioned above, the raw data captured by the camera 122 and microphone 124 may be processed at the wearable device 102 in a way that protects the privacy of the participants in the interaction. For example, the wearable device 102 may operate to extract emotional data from the images collected by the camera 122 so that the raw image data does not need to be transmitted to the analysis server 114. Similarly, the wearable 102 may operate to extract content and tone information from the audio data collected by the microphone 124 so that the raw audio data does not need to transmitted to the analysis server 114. However, in other examples, this processing may occur at the analysis server or another secure network location with which the wearable device 102 can communicate wirelessly (e.g. using a wireless network).

The wearable device 102 may thus be provided with a data processing capability, e.g. comprises a processor 126, a memory 127 and software instructions stored on the memory, which when executed operate to perform the functions discussed herein. One of the functions may be an emotional state determination module, where the emotional state of a participant in the image data is derived from facial images, posture, head pose, etc. in any known manner.

Another of the functions may be a speech recognition and analysis module, which operates to extract content data from the collected audio data. Any suitable natural language processing tool may be used, e.g. NLTK or the like.

The wearable device 102 may further comprises a transceiver 128 configured to communicate in a wireless manner with the analysis server 114. The transceiver 128 may include a network module for communication over a wide area network (e.g. using WiFi or a cellular network). Alternatively, the transceiver 128 may communicate over a short range network (e.g. Bluetooth® or the like) with an intermediate device (e.g. a smartphone or tablet computer held by the second participant), where the intermediate device communicates with the analysis server 114. In one example, the processing capabilities of the wearable device 102 discussed above may be provided on the intermediate device, but this is not essential.

The transceiver 128 may be configured to receive data (e.g. software or firmware updates), e.g. from the analysis server 114 or some other control entity. FIG. 3 is a schematic diagram showing functional elements of the analysis module 115. These functional elements may be implemented as software modules in a conventional manner. The analysis module 115 comprises a data receiver 130 for receiving the collected data 112 and parsing or otherwise dividing it for further processing. In this example, the received collected data 112 comprises audio data and image data. The audio data is supplied to an audio analysis module 136, whereas the image data is supplied to an image analysis module 132. The image analysis module 132 is configured to extract emotional state data from the image data, e.g. using a classifier algorithm in a conventional manner. The audio data may also be used in this process, e.g. as a further input to the classifier. The audio analysis module 136 is configured to employ speech recognition technology to extract content data 138 from the collected audio data.

The audio analysis module 136 and the image analysis module 132 are depicted in dotted lines in FIG. 3, as their functions may be performed in the wearable device 102 (or the intermediate device) discussed above. In that case, the collected data 112 may already comprise the emotional state data 134 and content data 138.

The emotional state data 134 and content data 138 and tone data 140 are input to an analysis engine 142, which includes an algorithm 144 configured to evaluate, using the content data 138 and emotional state data 134 as inputs, an interaction score 148. The analysis engine 142 may also be configured to assign an interaction type 146 to the interaction, e.g. based on the content data 138.

The algorithm 144 may be a machine learning algorithm, such as a classifier trained using data in an interaction database 150. The interaction database 150 may include collected data for previous interactions that has been previously scored by human annotators. In some examples, the collected data 112 may be supplied to an annotation tool 152 for labelling, whereupon it can be used to supplement the data in the interaction database 150.

The output from the analysis engine 142 may include the emotional state data 134 and content data 138 together with the further information (e.g. interaction type 146 and interaction score 148) derived therefrom. This output may be used directly, e.g. as an interaction record 119, which may be a multi-dimensional representation of the evolution of the interaction, e.g. to allow the effectiveness of the interaction or the performance of the second participant to be assessed. The output may be displayed, e.g. in a graphical manner, on a suitable display. For example, the output may be displayed as time series of data points in each of the data sets.

The output from the analysis engine 142 may also be used to generate the feedback 116 discussed above. The analysis module 115 may include a feedback generator 154 that receives the output and supplies it as an input to a second algorithm 155 configured to generate the feedback 116. The second algorithm 155 may be trained using data from annotated (labelled) interaction records relating to earlier interactions, which are stored in an outcome database 156.

The data in the outcome database 156 may thus be the multi-dimensional representation of the evolution of a plurality of previous interactions (possibly between different participants but within a similar environment). The outcome data may further comprise an annotation that indicates a result of the interaction, in particular in relation to a predetermined objective (e.g. positive emotional, agreement to purchase, etc.). The second algorithm 155 may thus be used to evaluate the output data from the analysis engine 142 relative to the objective by comparing the interaction record 119 for the current interaction with previously obtained interaction records, e.g. to determine actions that may improve the performance relative to the objective. These actions form the basis for the feedback 116.

Additional information may be added to the outcome database 156, e.g. to update the second algorothm 155, by supplying the output data 118 from the analysis engine 142 to an annotation tool 158 where appropriate labels or tags are applied, e.g. to indicate one or more actual results arising from the interaction. Annotated data from the annotation tool 158 may be stored in the outcome database 156.

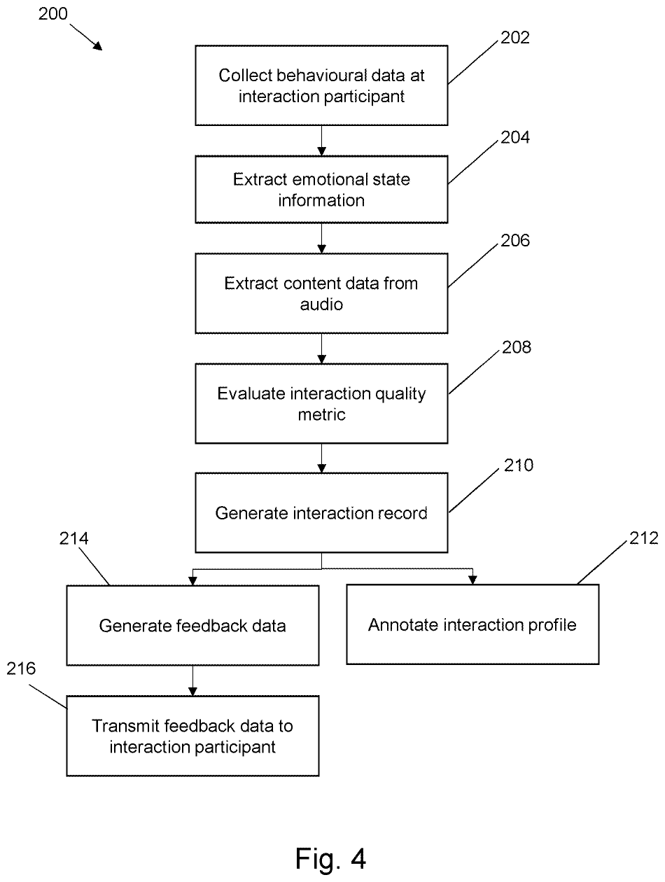

FIG. 4 is a flow diagram showing steps in a computer-implemented method 200 that is an embodiment of the invention. The method 200 may be carried out using a system such as that discussed above with respect to FIG. 1.

The method 200 begins with a step 202 of collecting behavioural data from one or more participants engaged in an interpersonal interaction. As discussed above, the behavioural data may comprise audio and image data, which may be collected at a wearable device on one or more of the participants, or at static devices that observe an area in which an interaction may take place.

The method 200 continues with a step 204 of extracting emotional state information from the collected behavioural information, e.g. from facial image data and/or information relating to posture, head pose, gaze direction, etc. of one or more participants. Step 204 may utilise behavioural data obtained concurrently from several sources to establish a participant's emotional state.

The method 200 continues with a step 206 of extracting content data from the collected audio data. Step 206 may be performed simultaneously or sequentially to step 204.

The method 200 continues with a step 208 of evaluating an interaction quality metric to obtain an interaction score for the interaction. The method may also include assigning an interaction type.

The method continues with a step 210 that takes the output from step 208 to generate an interaction record, in which the evolution with time of the various parameters discussed above (e.g. emotion, content, engagement, score) may be consolidated. This profile may be used to assess the performance of a participant in the interaction or to assist in their training.

The method may further comprise a step 212 of annotating the interaction record with an outcome of the interaction. The annotated profile from multiple interactions may be used as a training set for a model that operates to map an interaction profile to an desired objective. As discussed, such a model may be used to generate recommendations or suggestions for increasing the probability that an objective is achieved. Steps 214 and 216 utilise such a model.

The method may continue with a step 214 of generating feedback data using the various parameters discussed above (e.g. emotion, content, engagement, score) or the interaction record as an input. The method continues with a step 216 of transmitting the feedback data to the interaction participant. As mentioned above, the feedback data may comprise recommendations or suggestions for increasing the probability that an objective is achieved.

In the examples discussed above, the participant may be human or robot. The techniques of the invention may be particularly useful in scenarios where both human and robot participants are available. In observing the interaction profile for a plurality of interactions, the system may determine categorisations for interactions, i.e. to allow different types of interaction to be identified objectively. The system may be further configured to compare the performance of human and robot participants in each type of interaction. As a result, the system may be able to determine which types of interaction are better served by robot participants and which are better served by human participants. Moreover, the comparison data may assist in identifying areas in which improvement in a robot's ability would be most beneficial, e.g. to match or exceed human performance.

In a development of the ideas above, the system may also learn the preferences of other participants, e.g. through their previous interactions. For example, customers of a shop may agree to have a stored profile in the system. When entering the shop, the customer may be recognised by the behavioural data collection devices, whereupon any preferences associated with their profile may be used to influence how to handle their in-store interactions.

1. A system for collecting behavioural data during an interpersonal interaction, the system comprising:
a wearable device comprising:
a data collection device configured to capture behavioural data during the interpersonal interaction;
a microphone configured to capture audio data during the interpersonal interaction;
an analysis module arranged to:
extract emotional state information from the behavioural data;
extract content data from the audio data; and
evaluate, using the emotional state information and content data, an interaction quality metric to obtain an interaction score for the interpersonal interaction.

2. The system of claim 1, wherein the analysis module is further arranged to determine an interaction type for the interpersonal interaction based on the content data.

3. The system of claim 2, wherein the analysis module is further arranged to generate and store an interaction record for the interpersonal interaction, wherein the interaction record is a data structure that associates the interaction type and interaction score with an emotional state profile that is indicative of variation in the emotion state information during the interpersonal interaction.

4. The system of claim 3, wherein the interaction record data structure further comprises:
a plurality of content tags indicative of the content data; and/or
participant identification data.

5. (canceled)

6. The system of claim 5-1 further comprising a feedback generation module arranged to:
determine a recommendation for improving the interaction score for the interpersonal interaction;
generate a feedback instruction corresponding to the recommendation; and
output the feedback instruction.

7. The system of claim 6 further comprising a feedback communicator arranged to receive the feedback instruction from the analysis engine.

8. The system of claim 7, wherein the feedback communicator comprises a second wearable device.

9. The system of claim 1, wherein the data collection device comprises an imaging device configured to capture images of the interpersonal interaction, wherein the behavioural data comprises image data, and wherein the analysis module comprises:
an image analyser module arranged to extract emotional state information from the image data captured by the imaging device; and
a speech recognition module configured to extract the content data from the audio data captured by the microphone.

10. (canceled)

11. The system of claim 1, wherein the analysis module comprises an analysis engine configured to evaluate, using the emotional state information and content data, the interaction quality metric to obtain the interaction score.

12. The system of claim 11, wherein the analysis engine is located remotely from the wearable device, and wherein the wearable device further comprises a transceiver configured to wirelessly transmit the emotional state information and the content data to the analysis engine.

13. The system of claim 11, wherein the analysis engine is provided on the wearable device.

14. The system of claim 1 further comprising an intermediate computing device arranged in wireless communication with the wearable device,
wherein the analysis module is provided on the intermediate computing device, and
wherein the wearable device further comprises a transceiver configured to wirelessly communicate the behavioural data and audio data to the intermediate computing device.

15. The system of claim 1, wherein the wearable device comprises a processor and a memory having software instructions stored thereon, wherein the analysis module comprises one or more software modules which run by execution of the software instructions by the processor.

16. The system of claim 1, wherein the behavioural data relates to a first participant in the interpersonal interaction, and the wearable device is configured for a second participant in the interpersonal interaction.

17. The system of claim 16, wherein the second participant is a social robot.

18. A computer-implemented method of collecting behavioural data relating to an interpersonal interaction between a first participant and a second participant, the method comprising:
collecting behavioural data of the first participant using a wearable device worn by the second participant, wherein the behavioural data comprises an image data stream and an audio data stream captured during the interpersonal interaction;
extracting an emotional state information stream from the image data stream;
extracting a content data stream from the audio data; and
evaluating, using the emotional state information stream and content data stream, an interaction quality metric to obtain an interaction score for the interpersonal interaction.

19. The method of claim 18 further comprising:
determining an interaction type for the interpersonal interaction based on the content data; and
generating an interaction record for the interpersonal interaction, wherein the interaction record is a data structure that associates the interaction type and interaction score with an emotional state profile that is indicative of variation in the emotion state information during the interpersonal interaction.

20. The method of claim 19, wherein the interaction record further comprises any one or more of:
a plurality of content tags indicative of the content data; and
participant identification data.

21. (canceled)

22. The method of claim 18 further comprising:
determining a recommendation for improving the interaction score for the interpersonal interaction;
generating a feedback instruction corresponding to the recommendation; and
outputting the feedback instruction.

23. The method of claim 19 further comprising:
obtaining a first plurality of interaction records from multiple interpersonal interactions in which the second participant is a human;
obtaining a second plurality of interaction records from multiple interpersonal interactions in which the second participant is a social robot;
comparing the first plurality of interaction records with the second plurality of interaction records to determine a performance metric for the human and the social robot; and
selecting either the human or the social robot to participate in a future interaction based on the determined performance metric.
